(19) United States
(12) Patent Application Publication
(10) Pub. No.: US 2026/0165764 A1
(43) Pub. Date: Jun. 18, 2026

(54) MINIMALLY INVASIVE MEDICAL DEVICE FOR SAFE DELIVERY OF ELECTRICAL PULSES TO TARGET TISSUE PERCUTANEOUSLY USING A SINGLE NEEDLE AND A GROUNDING PAD

(71) Applicant: Georgia Tech Research Corporation, Atlanta, GA (US)

(72) Inventors: Vinay Deshmukh, Atlanta, GA (US); Rafael Davalos, Atlanta, GA (US)

(21) Appl. No.: 19/419,746

(22) Filed: Dec. 15, 2025

Related U.S. Application Data
(60) Provisional application No. 63/733,719, filed on Dec. 13, 2024.

(51) Int. Cl. A61B 18/00; A61B 17/00

(52) U.S. Cl. CPC A61B 18/00; A61B 2017/00867; A61B 2018/00184; A61B 2018/00577; A61B 2018/00767

(57) ABSTRACT

The present disclosure relates to systems and methods for the percutaneous delivery of electrical energy to tissue of a subject, and particularly to pulse field ablation systems and methods for applying electrical potential differences to ablate tissue. A pulse field ablation system includes a grounding electrode configured for percutaneous placement in a subject, a needle electrode configured to be positioned in proximity to the grounding electrode, and a controller configured to apply a potential difference between the grounding electrode and the needle electrode to ablate tissue in the subject.

### CROSS-REFERENCE TO RELATED APPLICATIONS

This application claims the benefit of U.S. Provisional Application Ser. No. 63/733,719, filed on 13 Dec. 2024, which is incorporated herein by reference in its entirety as if fully set forth below.

### GOVERNMENT LICENSE RIGHTS

This invention was made with government support under CA240476 and CA274439 awarded by the National Institutes of Health. The government has certain rights in this invention.

### FIELD OF INVENTION

The present disclosure relates to systems and methods for the percutaneous delivery of electrical energy to tissue of a subject, and more particularly to a pulse field ablation (PFA) system and method comprising a grounding electrode configured for percutaneous placement in a subject and a needle electrode in proximity to the grounding electrode for applying electrical potential differences to ablate tissue.

### BACKGROUND

PFA is a non-thermal ablation modality that delivers short pulses of electric current to permanently destabilize the plasma membrane through increasing the transmembrane potential. PFA can be further delineated into reversible (RE) and irreversible electroporation (IRE). The term irreversible refers to the cells' inability to maintain viability and homeostasis after applying successive high current pulses. After the application of IRE, cell death occurs in various forms including necrosis, apoptosis, necroptosis, and pyroptosis. Targeted cell death through IRE enables safe, cost effective, and highly localized treatments to unwanted cells.

One of the characteristic features of IRE is the threshold energy that must be applied for the cell to become irreversible. However, this threshold has several variables, namely, the waveform that is being used. These waveforms include the shape (square, sinusoidal, triangular, etc.), off-time, frequency, current, voltage peak, and direction. Since its inception, IRE waveforms have been modified to conform to the unique clinical application and devices being used. One such modification to the waveform was the introduction of High-Frequency Irreversible Electroporation (H-Fire). H-fire was designed to target and mitigate muscle contractions that are caused by nerve excitation which occurs when low intensity stray current (electric fields) perfuse through tissue. These extraneous electric fields occur at some distance away from the region of irreversibility, and don't cause long term tissue damage. The excitation of nerves is contingent on the duration and intensity of the applied electric field.

Successful ablation procedures involve the waveform working in conjunction with the mechanical apparatus, the targeted anatomy, and safety of the patient. Clinical applications of PFA utilize specifically designed electrodes for each procedure. While these devices are designed with physicians in mind, targeted design optimizations could improve ablation predictability, improve clinician confidence, and improve widespread adoption of PFA for untapped medical procedures.

In oncology, radio-frequency (RF) and PFA are the two most common ablation methods. PFA has been found to increase the macrophage migration inhibitory factor, which may improve the reparative process and may result in the shrinkage of the targeted ablation area. There are three primary probes that are used for PFA based procedures. The first and most widely used, is the multi-electrode method where the clinician places several electrodes around the legion volume and ablates between the electrodes. The second probe design is a single insertion bipolar probe where the anode and cathode reside on the same needle. Finally, the third procedure is the combination of a single needle and a grounding pad—where there is a single needle inserted into the body and a large grounding pad that is placed externally on the skin. Each design has its pros and cons; however, the multielectrode design is used most due to its ablation size and low arching potential.

Current single needle grounding pad systems place the grounding pad somewhere on the skin, typically by the ankle or back. This configuration can result in muscle stimulation and nerve excitation due to the distance between the grounding pad and the needle electrode. Additionally, the skin acts as a resistor or insulator, which can affect the efficiency of the electric field distribution and may limit the effectiveness of the ablation procedure.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a pulse field ablation system can be provided. The system can include a grounding electrode configured for percutaneous placement in a subject. The system can include a needle electrode configured to be positioned in proximity to the grounding electrode. The system can include a controller configured to apply a potential difference between the grounding electrode and the needle electrode to ablate tissue in the subject.

According to another aspect of the present disclosure, a method of performing pulse field ablations can be provided. The method can include placing a grounding electrode percutaneously in a subject. The method can include positioning a needle electrode in proximity to the grounding electrode. The method can include applying an electric potential difference between the grounding electrode and the needle electrode to ablate tissue in the subject.

According to another aspect of the present disclosure, a pulse field ablation device can be provided. The device can include a catheter having a lumen. The device can include a grounding electrode disposed within the lumen in a contracted state and configured to transition to an expanded state when ejected from the catheter percutaneously in a subject. The device can include a needle electrode configured to be positioned in proximity to the grounding electrode when the grounding electrode is in the expanded state.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF FIGURES

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 illustrates a PFA device having a grounding electrode and needle electrode, according to examples of the disclosed technology.

FIG. 2 illustrates the PFA device of FIG. 1, according to examples of the disclosed technology.

FIG. 3 illustrates the PFA device of FIG. 1, according to examples of the disclosed technology.

FIG. 4 illustrates a grounding electrode having a support and hollow aperture, according to examples of the disclosed technology.

FIG. 5 illustrates the grounding electrode device of FIG. 4, according to examples of the disclosed technology.

FIG. 6 illustrates the grounding electrode device of FIG. 4, according to examples of the disclosed technology.

FIG. 7 is a flow diagram for a method for performing PFA, according to examples of the disclosed technology.

FIG. 8 is a flow diagram for a method for performing PFA, according to examples of the disclosed technology.

FIG. 9 is a flow diagram for a method for performing PFA, according to examples of the disclosed technology.

FIG. 10 is a flow diagram for a method for performing PFA, according to examples of the disclosed technology.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

Pulse field ablation (PFA) represents a non-thermal ablation modality that delivers short pulses of electric current to permanently destabilize cellular plasma membranes through increasing transmembrane potential. PFA can be further delineated into reversible (RE) and irreversible electroporation (IRE). The term “irreversible” distinguishes this technique from reversible electroporation, as cells lose the ability to maintain viability and homeostasis after application of successive high current pulses. Following IRE treatment, cell death may occur through various mechanisms including necrosis, apoptosis, necroptosis, and pyroptosis. Targeted cell death through IRE provides advantages including safe, cost-effective, and highly localized treatments to unwanted cells. PFA also includes tissue electroporation, which can be defined as the use of RE for molecular medicine. Tissue electroporation can be used to introduce genes, drugs, or many other macromolecules into cells. Tissue electroporation includes electrogenetherapy and gene transfer.

PFA systems typically employ specifically designed electrodes for clinical applications, including multi-probe configurations, single bipolar probes, or probe-with-grounding-pad arrangements. Each electrode configuration presents distinct advantages and limitations regarding ablation size, placement accuracy, muscle stimulation, and procedural complexity. Conventional PFA systems may place grounding pads externally on the skin surface, such as near the ankle, which can result in increased muscle stimulation due to the extended distance between electrodes and the passage of electrical current through intervening tissues.

The disclosed technology addresses limitations of conventional PFA systems by providing improved electrode configurations and deployment methods. The systems and methods described herein may reduce muscle stimulation, improve ablation symmetry, and enhance placement ease compared to existing approaches. The technology may enable percutaneous placement of grounding electrodes in proximity to target tissues, potentially reducing the electrical path length and associated side effects while maintaining effective tissue ablation capabilities. The embodiments disclosed herein, however, are not limited to IRE systems; rather, as those skilled in the art would understand, various embodiments of the present disclosure can be utilized for any system in which it is desirable to apply electric potential between two or more electrodes inside the body of a subject.

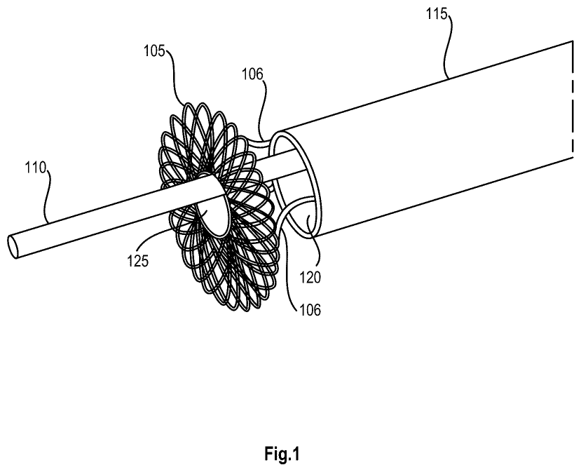

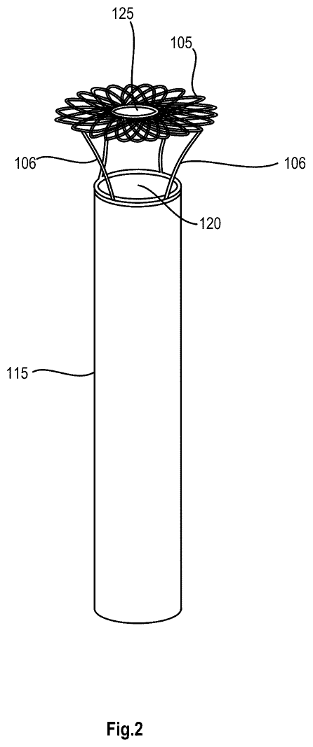

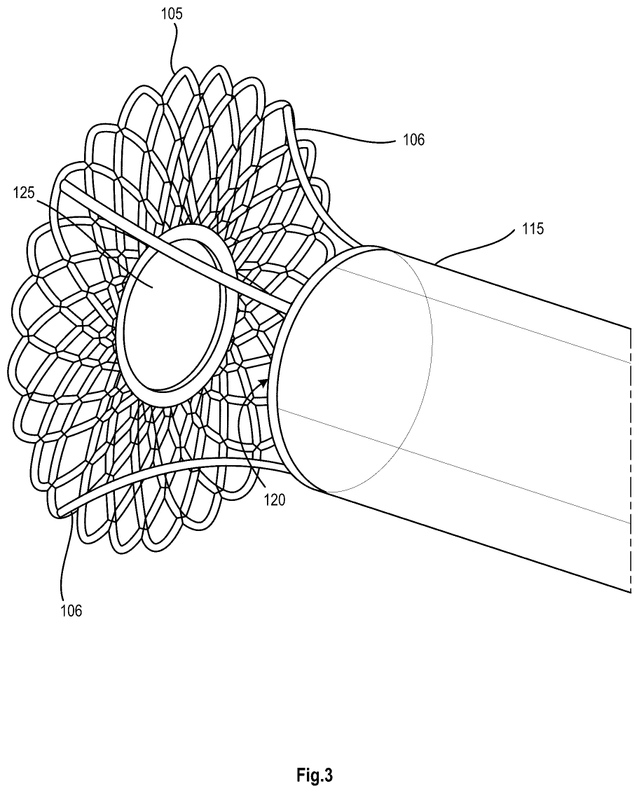

Referring to FIGS. 1-3, a pulse field ablation system may include a grounding electrode 105 configured for percutaneous placement in a subject. The grounding electrode 105 may comprise a substantially circular center portion and a plurality of leaflets circumferentially disposed around the center portion. In some cases, the grounding electrode 105 may be formed from a shape memory material that enables the grounding electrode 105 to transition between contracted and expanded configurations. The shape memory material may be nitinol, which provides the grounding electrode 105 with the ability to return to a predetermined shape when deployed from a catheter 115.

The grounding electrode 105 may be supported by a support 106 that maintains the structural integrity of the grounding electrode 105 during deployment and operation. The support 106 may extend from the grounding electrode 105 and provide a connection point for electrical and mechanical coupling to the catheter 115. The support 106 may facilitate advancement and retraction of the grounding electrode 105 through a lumen 120 of the catheter 115.

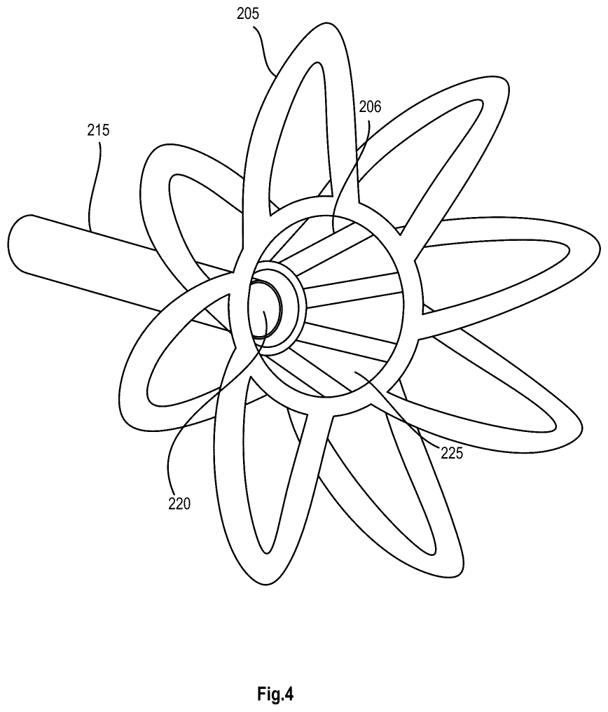

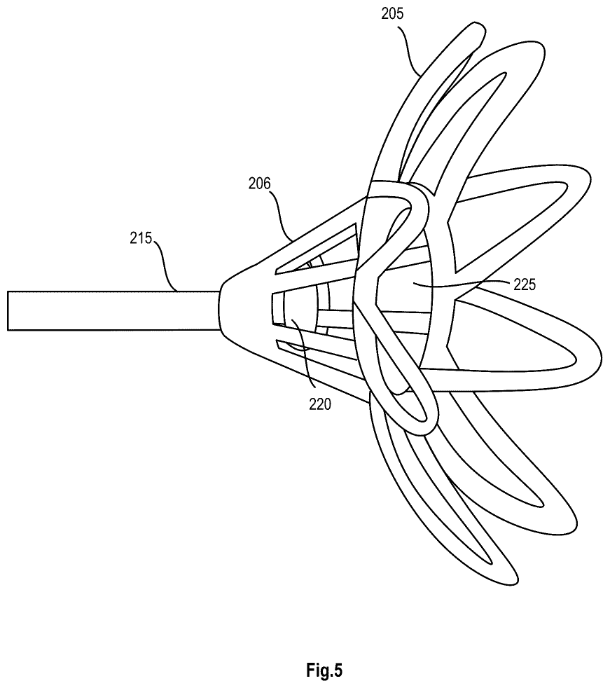

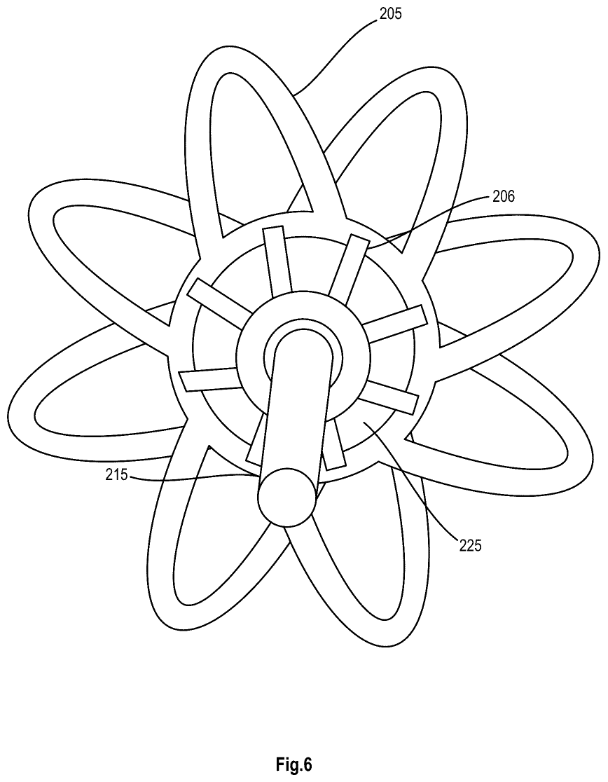

With reference to FIGS. 4-6, an alternative configuration of a grounding electrode 205 may be provided. The grounding electrode 205 may comprise a support 206 that forms a substantially circular center portion with a plurality of leaflets extending radially outward in a flower-like or star-like pattern. In some cases, the grounding electrode 205 may include eight leaflets, though other configurations with varying numbers of leaflets may be employed. Each leaflet may have an elongated, petal-shaped geometry that tapers from a wider base near the support 206 to a narrower distal end.

The grounding electrode 105, 205 may be configured in various geometrical forms to accommodate different clinical applications and anatomical requirements. In some cases, the grounding electrode 105, 205 may be configured as a mesh form that provides distributed electrical contact across a tissue surface. The mesh configuration may allow for flexibility and conformability to irregular tissue surfaces while maintaining electrical conductivity. Alternatively, the grounding electrode 105, 205 may be configured as a ring form that provides circumferential electrical contact around a target area. The grounding electrode 105, 205 may also be configured as a solid pad with various pad-like geometries, including circular, oval, rectangular, or other shapes suitable for specific anatomical locations. In embodiments where the grounding electrode 105, 205 is configured as a solid pad (e.g., a saline soaked gauze), the PFA system can be configured such that upon deployment, the needle electrode 110 pierces through a portion of the grounding electrode 105, 205. In such embodiments, a portion of the needle electrode 110 making contact with the grounding pad can include an insulative layer between the electrode and the grounding pad 105, 205 to prevent shorting.

The material composition of the grounding electrode 105, 205 may vary depending on the desired mechanical and electrical properties. In some cases, the grounding electrode 105, 205 may comprise nitinol with different weave patterns, gauge thicknesses, and compositions to achieve specific deployment characteristics and electrical conductivity. The weave patterns may include braided configurations, mesh patterns, or other interlaced structures that provide both flexibility and strength. The gauge thickness may be selected to balance mechanical properties with deliverability through the catheter 115, 215.

As an alternative to shape memory materials, the grounding electrode 105, 205 may comprise medical grade silicone lined with conductive wires. This configuration may provide a flexible substrate that conforms to tissue surfaces while maintaining electrical conductivity through the embedded conductive wires. The medical grade silicone may offer biocompatibility and flexibility, while the conductive wires may be arranged in patterns that optimize electrical field distribution across the grounding electrode 105, 205 surface.

The grounding electrode 105, 205 may be disposed within the lumen 120, 220 of the catheter 115, 215 in a contracted state during delivery to the target site. In the contracted state, the grounding electrode 105, 205 may be compressed or folded to fit within the confines of the lumen 120, 220, allowing for percutaneous insertion through small incisions or natural body openings. Upon deployment from the catheter 115, 215, the grounding electrode 105, 205 may transition to an expanded state where the leaflets extend outward to provide a larger surface area for electrical contact with tissue.

Referring to FIG. 1, a needle electrode 110 may be configured to be positioned in proximity to the grounding electrode 105. The needle electrode 110 may be positioned to extend through an aperture 125 of the grounding electrode 105, allowing for precise placement of the needle electrode 110 relative to the grounding electrode 105. In some cases, the needle electrode 110 may be configured to be disposed within the lumen 120 of the catheter 115, enabling delivery of the needle electrode 110 through the same catheter 115 used for grounding electrode 105 deployment.

The needle electrode 110 may comprise various distal end configurations to accommodate different clinical applications and procedural requirements. In some cases, the needle electrode 110 may have a solid tip that provides a sharp point for tissue penetration while maintaining structural integrity during insertion. The solid tip configuration may be suitable for standard ablation procedures where tissue penetration is the primary objective.

For drug delivery applications, the needle electrode 110 may have a hollow tip that enables delivery of therapeutic agents directly to the target tissue. The hollow tip may be connected to a drug delivery system that allows controlled infusion of medications, contrast agents, or other therapeutic substances during or after the PFA procedure. The hollow tip configuration may facilitate combination therapies where both electrical ablation and pharmacological treatment are desired.

In some cases, the needle electrode 110 may have a perforated tip with multiple holes distributed along the distal end. The perforated tip configuration may provide enhanced drug delivery capabilities by creating multiple exit points for therapeutic agents. The multiple holes may allow for more uniform distribution of delivered substances throughout the target tissue volume, potentially improving treatment efficacy compared to single-port delivery systems.

The needle electrode 110 may comprise one or more tines extending from the distal end to enhance tissue engagement and electrical contact. The tines may extend radially outward from the needle electrode 110 tip, creating multiple contact points with the surrounding tissue. This configuration may improve electrical field distribution and may provide more secure anchoring of the needle electrode 110 within the target tissue during the PFA procedure.

For procedures requiring thermal management, the needle electrode 110 may be water-cooled to manage thermal effects during operation. The water-cooled configuration may include internal channels or lumens within the needle electrode 110 that allow circulation of cooling fluid. The cooling system may help maintain optimal temperatures at the electrode-tissue interface and may prevent excessive heating that could compromise treatment effectiveness or cause unintended thermal damage.

The needle electrode 110 may be constructed from various conductive materials selected for their electrical, mechanical, and biocompatibility properties. In some cases, the needle electrode 110 may be made from platinum, which provides excellent corrosion resistance and biocompatibility. Alternatively, the needle electrode 110 may be made from aluminum, stainless steel, or gold, each offering different combinations of conductivity, strength, and cost considerations. The material selection may be tailored to specific procedural requirements and performance objectives.

In some cases, the system may comprise a plurality of needle electrodes instead of a single needle electrode 110. The plurality of needle electrodes may be positioned in proximity to the grounding electrode 105 to create multiple ablation zones or to treat larger tissue volumes. The multiple needle electrodes may be deployed sequentially or simultaneously, depending on the treatment protocol and the specific clinical application. Each needle electrode in the plurality may have the same or different tip configurations, materials, and dimensions to optimize treatment outcomes for complex anatomical targets.

Referring to FIGS. 1-3, a catheter 115 may be provided to facilitate percutaneous delivery of the grounding electrode 105 and needle electrode 110 to target sites within a subject. The catheter 115 may comprise a tubular structure that enables minimally invasive access through small incisions or natural body openings. In some cases, the catheter 115 may be constructed from polycarbonate extrusion material, which provides a combination of flexibility, strength, and biocompatibility suitable for medical applications. The polycarbonate extrusion material may offer transparency that allows visualization of internal components during deployment procedures.

The catheter 115 may include a lumen 120 that extends along the length of the catheter 115 to accommodate the grounding electrode 105 and associated components. The lumen 120 may be sized to house the grounding electrode 105 in a contracted state while providing sufficient space for smooth advancement and retraction of the grounding electrode 105 during deployment procedures. In some cases, the lumen 120 may have a circular cross-sectional geometry, though other cross-sectional shapes may be employed depending on the specific configuration of the grounding electrode 105 and procedural requirements.

With continued reference to FIGS. 1-3, the grounding electrode 105 may be configured to be disposed within the lumen 120 of the catheter 115 in the contracted state. In the contracted state, the leaflets of the grounding electrode 105 may be compressed or folded to reduce the overall diameter of the grounding electrode 105, allowing the grounding electrode 105 to fit within the confines of the lumen 120. The grounding electrode 105 may be configured to transition to an expanded state when ejected from the catheter 115, where the shape memory properties of the grounding electrode 105 cause the leaflets to extend outward to their predetermined configuration.

As shown in FIGS. 4-6, the catheter 215 may similarly house the grounding electrode 205 within a lumen 220. The lumen 220 may accommodate the grounding electrode 205 in a contracted state, where the leaflets of the grounding electrode 205 are compressed to fit within the internal dimensions of the lumen 220. The catheter 215 may be constructed from the same polycarbonate extrusion material as the catheter 115, providing consistent material properties and performance characteristics across different grounding electrode configurations.

The needle electrode 110 may be configured to be disposed within the lumen 120 of the catheter 115, enabling delivery of both the grounding electrode 105 and the needle electrode 110 through a single catheter system. In some cases, the lumen 120 may be sized to accommodate both components simultaneously, with the needle electrode 110 positioned to extend through the aperture 125 of the grounding electrode 105 during deployment. This configuration may allow for coordinated deployment of both electrodes while maintaining their relative positioning throughout the procedure.

In some embodiments, the PFA device can be a single-use or multi-use device. Multi-use devices allow for deployment and retraction from the lumen multiple times.

The grounding electrode 105, 205 may be deployed using a hollow metal shaft connected to the grounding electrode 105, 205 that extends past a covering sheath within the catheter 115, 215. The hollow metal shaft may provide structural support for the grounding electrode 105, 205 during advancement through the lumen 120, 220 and may facilitate controlled deployment at the target site. The covering sheath may protect the grounding electrode 105, 205 during transit through the catheter 115, 215 and may be retracted to allow expansion of the grounding electrode 105, 205 upon reaching the desired location.

In configurations where the grounding electrode 105, 205 comprises a solid pad geometry without a central aperture, the grounding electrode 105, 205 may be backloaded into the catheter 115, 215. The backloading approach may involve inserting the grounding electrode 105, 205 into the lumen 120, 220 from the proximal end of the catheter 115, 215, allowing for independent deployment of the grounding electrode 105, 205 and needle electrode 110. This configuration may provide greater flexibility in electrode positioning and may enable treatment of multiple target sites with a single grounding electrode 105, 205 placement.

Referring to FIG. 1, the grounding electrode 105 may comprise an aperture 125 extending through the center portion of the grounding electrode 105. The aperture 125 may be configured to receive the needle electrode 110, allowing the needle electrode 110 to extend through the center of the grounding electrode 105 during deployment and operation. The aperture 125 may be sized to accommodate the outer diameter of the needle electrode 110 while maintaining proper electrical isolation between the grounding electrode 105 and the needle electrode 110.

The aperture 125 may be dimensioned to provide a close fit with the needle electrode 110 to minimize lateral movement while allowing smooth insertion and advancement of the needle electrode 110 through the grounding electrode 105. In some cases, the aperture 125 may include a slight clearance to account for manufacturing tolerances and to facilitate easy insertion of the needle electrode 110 during clinical procedures. The aperture 125 may extend completely through the thickness of the grounding electrode 105, creating an unobstructed pathway for the needle electrode 110.

With reference to FIG. 4, the grounding electrode 205 may comprise a hollow aperture 225 extending through the center portion of the grounding electrode 205. The hollow aperture 225 may provide a larger opening compared to standard aperture configurations, potentially accommodating needle electrodes with larger diameters or multiple needle electrodes simultaneously. The hollow aperture 225 may be formed as an integral part of the support 206, creating a continuous opening that extends through the central region of the grounding electrode 205.

The hollow aperture 225 may be configured to maintain structural integrity of the grounding electrode 205 while providing sufficient space for needle electrode insertion. In some cases, the hollow aperture 225 may include reinforcement features around the perimeter to prevent deformation or tearing during needle electrode insertion. The hollow aperture 225 may be sized to accommodate various needle electrode configurations, including those with tines, perforated tips, or cooling systems that may require additional clearance.

In configurations where the grounding electrode 105, 205 comprises a solid aperture, the needle electrode 110 may be inserted separately from the grounding electrode 105, 205 using independent insertion. The solid aperture configuration may provide a completely closed grounding electrode surface without any central opening, requiring the needle electrode 110 to be positioned through a separate insertion pathway. This independent insertion approach may allow for greater flexibility in needle electrode positioning relative to the grounding electrode 105, 205.

The solid aperture configuration may enable the physician to target multiple treatment sites with a single grounding electrode 105, 205 placement by repositioning the needle electrode 110 to different locations while maintaining the grounding electrode 105, 205 in a fixed position. The independent insertion may also allow for different approach angles for the needle electrode 110, potentially improving access to anatomically challenging target sites while maintaining optimal grounding electrode 105, 205 contact with tissue surfaces.

In system configurations, the grounding electrode 105, 205 may comprise the aperture 125 or hollow aperture 225 extending through the center portion, and the needle electrode 110 may be configured to extend through the aperture 125 or hollow aperture 225. The system may enable coordinated deployment where both electrodes are delivered through the same catheter 115, 215 while maintaining their relative positioning throughout the procedure.

In device configurations, the grounding electrode 105, 205 may comprise the aperture 125 or hollow aperture 225 extending through the center portion, and the needle electrode 110 may be configured to extend through the aperture 125 or hollow aperture 225 when the grounding electrode 105, 205 is in the expanded state. The device may enable the needle electrode 110 to be configured to be ejected from the catheter 115, 215 through the aperture 125 or hollow aperture 225 of the grounding electrode 105, 205, providing a streamlined deployment sequence for both electrodes.

The grounding electrode 105, 205 may be configured to undergo a controlled transition between contracted and expanded states to facilitate percutaneous delivery and deployment. In the contracted state, the grounding electrode 105, 205 may be compressed or folded to fit within the dimensional constraints of the catheter lumen 120, 220. The contracted configuration may reduce the overall profile of the grounding electrode 105, 205, enabling passage through small-diameter catheters and minimally invasive access to target anatomical sites.

During the contracted state, the leaflets of the grounding electrode 105, 205 may be compressed radially inward toward the central axis of the catheter 115, 215. The shape memory material properties of the grounding electrode 105, 205 may allow the leaflets to be deformed from their natural expanded configuration without permanent structural damage. The contracted state may be maintained by the constraining forces exerted by the catheter lumen 120, 220 walls, which prevent the grounding electrode 105, 205 from returning to the expanded configuration until deployment occurs.

The ejection process from the catheter 115, 215 may involve advancing the grounding electrode 105, 205 distally through the lumen 120, 220 until the grounding electrode 105, 205 exits the distal end of the catheter 115, 215. In some cases, the ejection may be facilitated by a pusher mechanism or deployment system that provides controlled advancement of the grounding electrode 105, 205 through the catheter 115, 215. The ejection process may be performed under imaging guidance to ensure accurate placement of the grounding electrode 105, 205 at the target tissue site.

Referring to FIG. 4, upon ejection from the catheter 215, the grounding electrode 205 may transition to an expanded state where the shape memory properties cause the leaflets to extend outward from the support 206. The expanded state may be achieved automatically as the constraining forces of the catheter lumen 220 are removed, allowing the grounding electrode 205 to return to the predetermined expanded configuration. The transition to the expanded state may occur rapidly upon deployment, providing immediate tissue contact and electrical connectivity.

In the expanded state, the grounding electrode 205 may form a flower-like or star-like pattern with the leaflets extending radially outward from the central support 206. The flower-like pattern may provide distributed electrical contact across a tissue surface while maintaining structural stability during the PFA procedure. The star-like configuration may offer enhanced conformability to irregular tissue surfaces, allowing the grounding electrode 205 to adapt to anatomical variations and maintain consistent electrical contact.

The expanded configuration may provide a larger surface area for electrical contact compared to the contracted state, potentially improving current distribution and reducing current density at individual contact points. The leaflets in the expanded state may be configured to apply gentle pressure against tissue surfaces, ensuring reliable electrical contact while minimizing tissue trauma. The expanded state may be maintained throughout the PFA procedure, providing stable electrode positioning and consistent electrical performance.

In system configurations, the grounding electrode 105, 205 may be configured to transition to an expanded state when ejected from the catheter 115, 215. The system may enable controlled deployment where the transition from contracted to expanded states occurs in a predictable manner, allowing for precise positioning of the grounding electrode 105, 205 at the target site.

In device configurations, the grounding electrode 105, 205 may be configured to transition to an expanded state when ejected from the catheter 115, 215 percutaneously in a subject. The device may provide a complete assembly where the grounding electrode 105, 205 is pre-loaded within the catheter 115, 215 in the contracted state and ready for deployment through percutaneous access routes to reach internal anatomical targets.

A controller may be configured to apply a potential difference between the grounding electrode and the needle electrode to ablate tissue in the subject. The controller may comprise electronic circuitry and software components that generate and regulate electrical signals delivered to the electrodes during PFA procedures. The controller may be configured to produce various waveform types, including square wave, sinusoidal, triangular, and other pulse configurations suitable for different clinical applications and treatment protocols.

The controller may be configured to operate with various PFA parameters to optimize treatment outcomes for specific tissue types and clinical objectives. The controller may provide adjustable on-time settings that determine the duration of electrical pulse delivery, with typical ranges from microseconds to milliseconds depending on the treatment protocol. Similarly, the controller may provide adjustable off-time settings that control the interval between pulses, allowing for tissue recovery and thermal management during multi-pulse sequences.

Voltage levels generated by the controller may be adjustable across a range suitable for different tissue types and electrode configurations. The controller may provide voltage outputs ranging from a few volts (e.g., less than 10 V) to hundreds of volts to several thousand volts, with precise control to achieve desired electric field strengths at the target tissue. Current levels may also be regulated by the controller to maintain consistent electrical delivery while accounting for tissue impedance variations and electrode contact conditions.

The controller may be configured to work with any PFA waveforms, include high-frequency irreversible electroporation (H-FIRE) waveforms in addition to standard IRE protocols. H-FIRE waveforms may comprise biphasic pulse sequences with shorter pulse durations and higher frequencies compared to conventional IRE, potentially reducing muscle contractions and nerve excitation during treatment. The controller may generate H-FIRE waveforms with alternating polarity phases to minimize charge accumulation and reduce unwanted physiological effects.

In some cases, the controller may be configured for reversible electroporation (RE) applications where temporary membrane permeabilization is desired rather than permanent cell death. For RE applications, the controller may generate lower voltage pulses with specific timing parameters that create transient pores in cell membranes without causing irreversible damage. The RE configuration may be particularly useful for drug delivery applications where enhanced cellular uptake is desired while preserving cell viability.

The controller may be configured to cyclically apply electrical potential differences between the grounding electrode and multiple needle electrodes when a plurality of needle electrodes is employed. The cyclic application may involve sequential activation of individual needle electrodes or groups of needle electrodes, allowing for controlled treatment of multiple tissue regions while maintaining optimal current distribution. The controller may coordinate the timing and sequencing of electrical delivery to each needle electrode to prevent interference and ensure consistent treatment parameters across all target sites.

The system may be configured for robotically assisted surgery with motorized deployment of the grounding electrode and needle electrode. In robotically assisted configurations, the controller may interface with robotic control systems to coordinate electrode deployment with electrical parameter delivery. The motorized deployment may provide precise positioning control and may enable automated sequences for electrode advancement, positioning, and retraction. The robotic integration may allow for enhanced precision and repeatability compared to manual deployment methods, particularly in anatomically challenging locations or when treating multiple target sites during a single procedure.

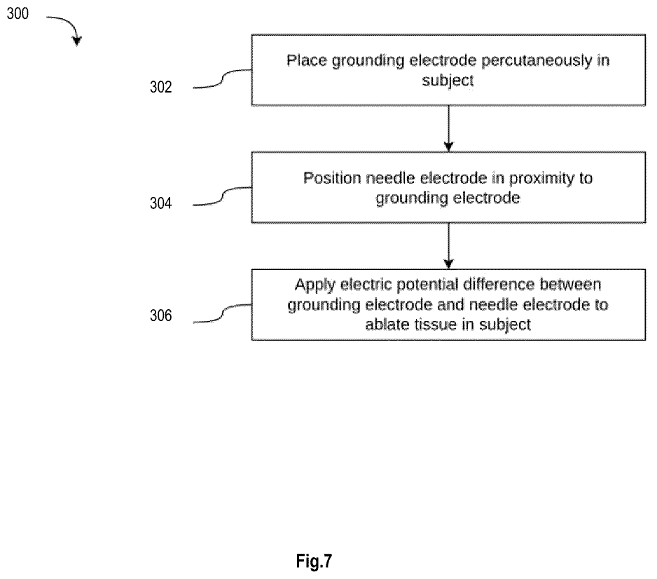

Referring to FIG. 7, a method 300 of performing pulse field ablations may be provided to enable percutaneous treatment of target tissues within a subject. The method 300 may comprise a series of sequential steps that facilitate controlled delivery of electrical energy to achieve tissue ablation while minimizing invasiveness and procedural complexity. The method 300 may be performed using the grounding electrode 105, 205 and needle electrode 110 configurations described herein, providing a systematic approach to PFA procedures.

The method 300 may begin with a step 302 of placing a grounding electrode percutaneously in a subject. The step 302 may involve percutaneous insertion of the catheter 115, 215 containing the grounding electrode 105, 205 in a contracted state through a small incision or natural body opening. The percutaneous placement may be performed under imaging guidance to ensure accurate positioning of the grounding electrode 105, 205 at the target anatomical site. The step 302 may enable minimally invasive access to internal organs and tissues while avoiding the need for open surgical procedures.

In some cases, placing the grounding electrode 105, 205 percutaneously in the subject may comprise placing the grounding electrode 105, 205 on a surface of tissue to be ablated. The surface placement may involve positioning the grounding electrode 105, 205 in direct contact with the external surface of an organ or tissue structure, such as the liver, kidney, or other target organs. The surface contact may provide optimal electrical coupling between the grounding electrode 105, 205 and the target tissue while minimizing the electrical path length and associated resistance. The surface placement may enable the grounding electrode 105, 205 to conform to the anatomical contours of the tissue surface, providing distributed electrical contact across the treatment area.

Alternatively, placing the grounding electrode 105, 205 percutaneously in the subject may comprise placing the grounding electrode 105, 205 in a conductive fluid filled cavity within the subject. The conductive fluid filled cavity may include anatomical spaces such as the peritoneal cavity, pleural cavity, or other body cavities that contain conductive biological fluids. The placement within a conductive fluid filled cavity may provide enhanced electrical coupling through the surrounding fluid medium, potentially improving current distribution and reducing impedance variations. The conductive fluid may serve as an electrical medium that facilitates current flow between the grounding electrode 105, 205 and surrounding tissues.

The method 300 may continue with a step 304 of positioning a needle electrode in proximity to the grounding electrode. The step 304 may involve advancing the needle electrode 110 through the aperture 125 or hollow aperture 225 of the grounding electrode 105, 205 when the grounding electrode 105, 205 is in the expanded state. The positioning may be performed with precision to ensure optimal spacing between the grounding electrode 105, 205 and the needle electrode 110 for effective electrical field generation. The step 304 may enable the needle electrode 110 to penetrate into the target tissue while maintaining proper electrical isolation from the grounding electrode 105, 205.

The positioning of the needle electrode 110 in proximity to the grounding electrode 105, 205 may be accomplished through various approaches depending on the specific electrode configuration employed. In configurations where the grounding electrode 105, 205 comprises the aperture 125 or hollow aperture 225, the needle electrode 110 may be inserted through the central opening to achieve coaxial alignment with the grounding electrode 105, 205. In configurations where the grounding electrode 105, 205 comprises a solid aperture, the needle electrode 110 may be positioned through independent insertion pathways while maintaining appropriate spacing relative to the grounding electrode 105, 205.

The method 300 may conclude with a step 306 of applying an electric potential difference between the grounding electrode and the needle electrode to ablate tissue in the subject. The step 306 may involve activation of the controller to generate electrical pulses with predetermined voltage, current, and timing parameters suitable for the specific treatment protocol. The electric potential difference may create an electrical field between the grounding electrode 105, 205 and the needle electrode 110 that induces PFA in the intervening tissue. The step 306 may result in irreversible membrane permeabilization and subsequent cell death in the target tissue region, achieving the desired ablation effect while preserving surrounding healthy tissues.

The application of the electric potential difference may be performed using various waveform configurations and parameter settings to optimize treatment outcomes for different tissue types and clinical objectives. The electrical delivery may comprise multiple pulses delivered in sequence, with controlled timing intervals between pulses to allow for tissue response and thermal management. The step 306 may be monitored in real-time to ensure consistent electrical delivery and to make adjustments to treatment parameters as needed based on tissue response and impedance measurements.

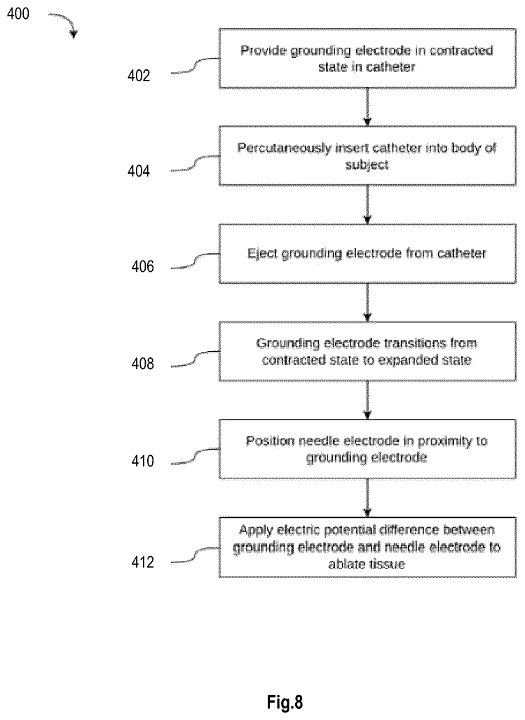

Referring to FIG. 8, a method 400 of performing PFA may utilize shape memory materials to facilitate controlled deployment of electrodes within a subject. The method 400 may provide enhanced deployment control through the use of materials that exhibit predictable shape transitions in response to temperature changes or removal of constraining forces. The method 400 may enable precise electrode positioning while maintaining the structural integrity of the grounding electrode throughout the deployment process.

The method 400 may begin with a step 402 of providing a grounding electrode in a contracted state in a catheter. The step 402 may involve loading the grounding electrode into the lumen of the catheter while the grounding electrode is compressed or folded to fit within the dimensional constraints of the catheter. The grounding electrode may comprise a shape memory material that allows the grounding electrode to be deformed from a natural expanded configuration without permanent structural damage. The contracted state may be maintained by the constraining forces exerted by the catheter walls, which prevent the grounding electrode from returning to the expanded configuration during transit through the catheter.

The shape memory material may be nitinol, which provides the grounding electrode with the ability to return to a predetermined shape when the constraining forces are removed. Nitinol may offer advantages including biocompatibility, corrosion resistance, and predictable shape recovery characteristics that make the material suitable for medical device applications. The nitinol composition may be selected to provide specific transformation temperatures and mechanical properties that optimize deployment performance for PFA procedures. The nitinol may be processed and heat-treated to establish the desired expanded configuration as the memorized shape, ensuring consistent deployment geometry across multiple uses.

The method 400 may continue with a step 404 of percutaneously inserting the catheter into a body of a subject. The step 404 may involve creating a small incision or accessing a natural body opening to introduce the catheter containing the grounding electrode in the contracted state. The percutaneous insertion may be performed under imaging guidance to ensure accurate navigation to the target anatomical site. The step 404 may enable minimally invasive access to internal organs and tissues while maintaining the grounding electrode in the contracted state during transit through the catheter.

Following the percutaneous insertion, the method 400 may proceed to a step 406 of ejecting the grounding electrode from the catheter. The step 406 may involve advancing the grounding electrode distally through the lumen until the grounding electrode exits the distal end of the catheter at the target tissue site. The ejection process may be facilitated by a pusher mechanism or deployment system that provides controlled advancement of the grounding electrode through the catheter. The step 406 may be performed with precision to ensure accurate placement of the grounding electrode at the desired location relative to the target tissue.

The method 400 may then include a step 408 where the grounding electrode transitions from the contracted state to an expanded state upon ejection. The step 408 may occur automatically as the constraining forces of the catheter lumen are removed, allowing the shape memory properties of the nitinol to cause the grounding electrode to return to the predetermined expanded configuration. The transition to the expanded state may occur rapidly upon deployment, providing immediate structural stability and positioning for subsequent electrode operations. The expanded state may provide a larger surface area for electrical contact compared to the contracted state, potentially improving current distribution and reducing current density at individual contact points.

The method 400 may continue with a step 410 of positioning a needle electrode in proximity to the grounding electrode. The step 410 may be performed after the grounding electrode has fully transitioned to the expanded state, ensuring proper spatial relationships between the electrodes. The positioning may involve advancing the needle electrode through an aperture of the grounding electrode or positioning the needle electrode through independent insertion pathways depending on the specific electrode configuration employed. The step 410 may enable the needle electrode to penetrate into the target tissue while maintaining appropriate electrical isolation from the grounding electrode.

The method 400 may conclude with a step 412 of applying an electric potential difference between the grounding electrode and the needle electrode to ablate tissue. The step 412 may involve activation of a controller to generate electrical pulses with predetermined parameters suitable for the specific treatment protocol. The electric potential difference may create an electrical field between the grounding electrode and the needle electrode that induces PFA in the intervening tissue. The step 412 may result in irreversible membrane permeabilization and subsequent cell death in the target tissue region while the grounding electrode remains in the stable expanded configuration provided by the shape memory material properties.

The use of nitinol as the shape memory material may provide several advantages for the deployment method. Nitinol may exhibit superelastic properties that allow the grounding electrode to undergo significant deformation during the contracted state while maintaining the ability to return to the original expanded shape upon deployment. The superelastic behavior may enable the grounding electrode to accommodate the geometric constraints of the catheter lumen without permanent deformation or structural failure. The nitinol may also provide consistent deployment characteristics across multiple temperature ranges encountered in clinical environments.

The shape memory properties of nitinol may be tailored through specific alloy compositions and heat treatment processes to achieve desired deployment characteristics. The transformation temperature of the nitinol may be set below body temperature to ensure that the grounding electrode transitions to the expanded state upon deployment within the subject. The mechanical properties of the nitinol may be optimized to provide sufficient force for reliable expansion while maintaining flexibility for conforming to tissue surfaces. The nitinol composition may also be selected to provide appropriate electrical conductivity for effective current distribution during PFA procedures.

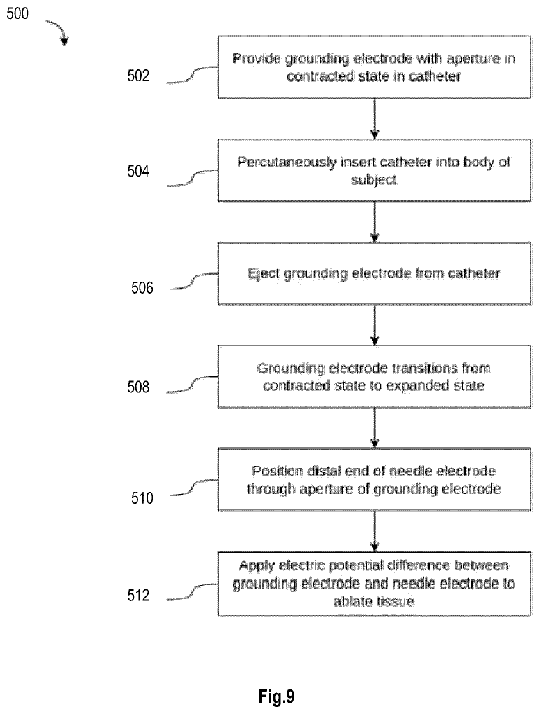

Referring to FIG. 9, a method 500 of performing PFA may utilize aperture-based positioning to achieve precise alignment between the grounding electrode and needle electrode during deployment. The method 500 may provide enhanced control over electrode positioning by utilizing a central aperture in the grounding electrode to guide needle electrode insertion and maintain coaxial alignment throughout the procedure. The method 500 may enable coordinated deployment of both electrodes through a single catheter system while ensuring proper spatial relationships for effective electrical field generation.

The method 500 may begin with a step 502 of providing a grounding electrode with an aperture in a contracted state in a catheter. The step 502 may involve loading the grounding electrode into the lumen of the catheter while the grounding electrode is compressed or folded to fit within the dimensional constraints of the catheter. The aperture may extend through the center portion of the grounding electrode, creating a continuous opening that remains accessible even when the grounding electrode is in the contracted state. The aperture may be dimensioned to accommodate the needle electrode while maintaining structural integrity of the grounding electrode during compression and deployment.

The grounding electrode may comprise a shape memory material that enables controlled transition between contracted and expanded configurations while maintaining the aperture geometry throughout the deployment process. The aperture may be formed as an integral part of the grounding electrode structure, ensuring that the opening remains properly sized and positioned relative to the leaflets or other structural elements of the grounding electrode. The contracted state may compress the leaflets radially inward while preserving the aperture dimensions to facilitate subsequent needle electrode insertion.

The method 500 may continue with a step 504 of percutaneously inserting the catheter into a body of a subject. The step 504 may involve creating a small incision or accessing a natural body opening to introduce the catheter containing the grounding electrode with the aperture in the contracted state. The percutaneous insertion may be performed under imaging guidance to ensure accurate navigation to the target anatomical site while maintaining the grounding electrode in the contracted configuration during transit through the catheter.

Following the percutaneous insertion, the method 500 may proceed to a step 506 of ejecting the grounding electrode from the catheter. The step 506 may involve advancing the grounding electrode distally through the lumen until the grounding electrode exits the distal end of the catheter at the target tissue site. The ejection process may be performed with precision to ensure accurate placement of the grounding electrode at the desired location relative to the target tissue while maintaining the integrity of the aperture for subsequent needle electrode positioning.

The method 500 may then include a step 508 where the grounding electrode transitions from the contracted state to an expanded state upon ejection. The step 508 may occur automatically as the constraining forces of the catheter lumen are removed, allowing the shape memory properties to cause the grounding electrode to return to the predetermined expanded configuration. The transition to the expanded state may position the leaflets in their final deployment geometry while maintaining the aperture in proper alignment for needle electrode insertion. The expanded state may provide structural stability and optimal tissue contact while preserving the aperture dimensions and accessibility.

The method 500 may continue with a step 510 of positioning a distal end of a needle electrode through the aperture of the grounding electrode. The step 510 may be performed after the grounding electrode has fully transitioned to the expanded state, ensuring that the aperture is properly positioned and accessible for needle electrode insertion. The positioning may involve advancing the needle electrode through the aperture to achieve coaxial alignment with the grounding electrode while maintaining appropriate electrical isolation between the electrodes. The distal end of the needle electrode may be guided through the aperture to penetrate into the target tissue at a predetermined depth relative to the grounding electrode position.

The aperture-based positioning approach may provide several advantages for electrode alignment and deployment control. The aperture may serve as a guide that maintains the needle electrode in proper alignment with the grounding electrode throughout the insertion process, reducing the risk of electrode misalignment or contact between the electrodes. The aperture may also provide a reference point for controlling the insertion depth of the needle electrode relative to the grounding electrode, enabling precise positioning of the needle electrode tip within the target tissue.

The method 500 may conclude with a step 512 of applying an electric potential difference between the grounding electrode and the needle electrode to ablate tissue. The step 512 may involve activation of a controller to generate electrical pulses with predetermined parameters suitable for the specific treatment protocol. The electric potential difference may create an electrical field between the grounding electrode and the needle electrode that induces PFA in the intervening tissue. The aperture-based positioning achieved in the step 510 may ensure optimal spacing and alignment between the electrodes, potentially improving the uniformity and predictability of the electrical field distribution during tissue ablation.

The positioning of the distal end of the needle electrode through the aperture may enable precise control over the electrode spacing and orientation during the PFA procedure. The aperture may maintain the needle electrode in a fixed relationship relative to the grounding electrode, preventing lateral movement or misalignment that could affect treatment outcomes. The coaxial alignment achieved through aperture-based positioning may provide symmetric electrical field distribution around the needle electrode, potentially improving ablation uniformity and reducing variations in treatment effectiveness across the target tissue volume.

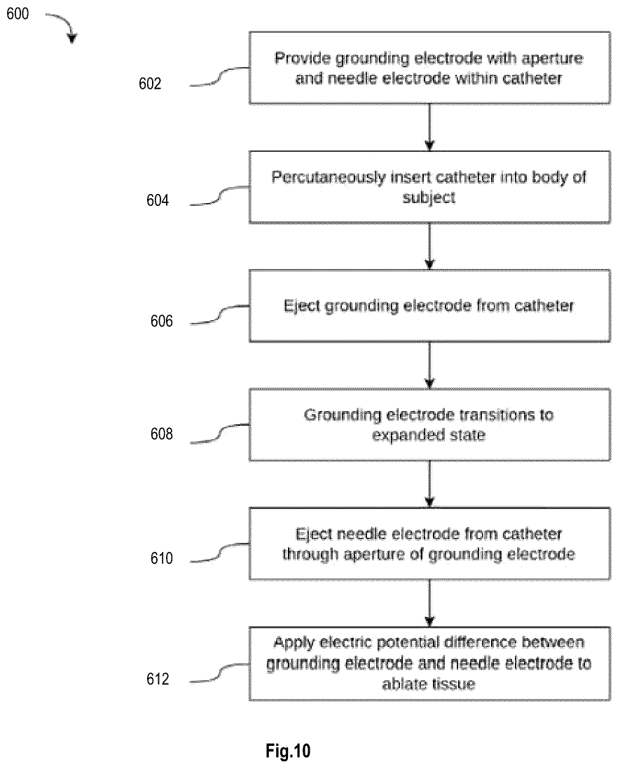

Referring to FIG. 10, a method 600 of performing PFA may utilize integrated catheter deployment where both the grounding electrode and needle electrode are provided within the catheter for coordinated delivery to target tissues. The method 600 may provide enhanced procedural efficiency by enabling simultaneous transport of both electrodes through a single catheter system while maintaining proper spatial relationships throughout the deployment sequence. The method 600 may facilitate streamlined electrode positioning and may reduce the complexity of multi-component PFA procedures.

The method 600 may begin with a step 602 of providing a grounding electrode with an aperture and a needle electrode within a catheter. The step 602 may involve loading both the grounding electrode and the needle electrode into the lumen of the catheter in a coordinated arrangement that enables sequential deployment while maintaining proper alignment between the electrodes. The grounding electrode may be positioned distally within the catheter relative to the needle electrode, allowing the grounding electrode to be deployed first to establish the reference position for subsequent needle electrode insertion. The aperture of the grounding electrode may be aligned with the needle electrode to facilitate passage of the needle electrode through the aperture during deployment.

The integrated catheter configuration may enable both electrodes to be transported to the target site through a single percutaneous access point, potentially reducing procedural invasiveness compared to systems requiring multiple insertion pathways. The catheter may be sized to accommodate both the grounding electrode in a contracted state and the needle electrode while maintaining sufficient space for smooth advancement and deployment of both components. The step 602 may establish the initial configuration for coordinated electrode delivery while ensuring that both electrodes remain properly positioned within the catheter during transit to the target anatomical site.

The method 600 may continue with a step 604 of percutaneously inserting the catheter into a body of a subject. The step 604 may involve creating a small incision or accessing a natural body opening to introduce the catheter containing both the grounding electrode and the needle electrode. The percutaneous insertion may be performed under imaging guidance to ensure accurate navigation to the target anatomical site while maintaining both electrodes in their respective positions within the catheter. The step 604 may enable minimally invasive access to internal organs and tissues while preserving the spatial relationships between the grounding electrode and needle electrode during catheter advancement.

Following the percutaneous insertion, the method 600 may proceed to a step 606 of ejecting the grounding electrode from the catheter. The step 606 may involve advancing the grounding electrode distally through the lumen until the grounding electrode exits the distal end of the catheter at the target tissue site. The ejection of the grounding electrode may be performed while the needle electrode remains within the catheter, allowing the grounding electrode to be positioned and stabilized before needle electrode deployment. The step 606 may ensure accurate placement of the grounding electrode at the desired location relative to the target tissue while maintaining the needle electrode in a ready position for subsequent deployment.

The method 600 may then include a step 608 where the grounding electrode transitions from a contracted state to an expanded state upon ejection. The step 608 may occur automatically as the constraining forces of the catheter lumen are removed, allowing the shape memory properties to cause the grounding electrode to return to the predetermined expanded configuration. The transition to the expanded state may position the grounding electrode in proper contact with the tissue surface while establishing the aperture in the correct orientation for needle electrode insertion. The expanded state may provide structural stability and optimal electrical contact while maintaining the aperture accessibility for the needle electrode that remains within the catheter.

The method 600 may continue with a step 610 of ejecting the needle electrode from the catheter through the aperture of the grounding electrode. The step 610 may be performed after the grounding electrode has fully transitioned to the expanded state and achieved stable positioning at the target site. The ejection of the needle electrode through the aperture may provide precise guidance for needle electrode insertion while maintaining coaxial alignment with the grounding electrode. The step 610 may enable the needle electrode to penetrate into the target tissue at a predetermined depth and orientation relative to the grounding electrode position, ensuring optimal spacing for electrical field generation.

The integrated deployment approach may provide several advantages for electrode positioning and procedural control. The sequential ejection of the grounding electrode followed by the needle electrode may enable the grounding electrode to serve as a positioning reference for the needle electrode, potentially improving alignment accuracy compared to independent insertion methods. The aperture of the grounding electrode may guide the needle electrode during ejection from the catheter, reducing the risk of electrode misalignment or contact between the electrodes during deployment.

The method 600 may conclude with a step 612 of applying an electric potential difference between the grounding electrode and the needle electrode to ablate tissue. The step 612 may involve activation of a controller to generate electrical pulses with predetermined parameters suitable for the specific treatment protocol. The electric potential difference may create an electrical field between the grounding electrode and the needle electrode that induces PFA in the intervening tissue. The coordinated deployment achieved through the integrated catheter method may ensure optimal electrode positioning and spacing, potentially improving the uniformity and predictability of the electrical field distribution during tissue ablation.

In device configurations utilizing the integrated catheter deployment approach, a PFA device may comprise a catheter having a lumen, a grounding electrode disposed within the lumen in a contracted state, and a needle electrode configured to be positioned in proximity to the grounding electrode when the grounding electrode is in the expanded state. The device may enable the needle electrode to be disposed within the lumen of the catheter and configured to be ejected from the catheter through the aperture of the grounding electrode. This device configuration may provide a complete assembly where both electrodes are pre-loaded within the catheter and ready for coordinated deployment through percutaneous access routes.

The device configuration may enable the needle electrode to be ejected from the catheter through the aperture of the grounding electrode after the grounding electrode has been deployed and transitioned to the expanded state. The aperture may serve as a guide that maintains proper alignment between the electrodes during the ejection process, ensuring that the needle electrode follows the predetermined insertion pathway established by the grounding electrode positioning. The device may provide consistent electrode spacing and orientation across multiple procedures, potentially improving treatment reproducibility and clinical outcomes.

The needle electrode may comprise insulation layers that prevent unwanted electrical contact with surrounding tissues during insertion and positioning procedures. The insulation layers may be formed from polyamide tubing that provides electrical isolation along the length of the needle electrode while maintaining flexibility for catheter delivery. In some cases, the insulation layers may comprise Microlumen materials that offer precise dimensional control and biocompatibility for medical device applications. The insulation may extend along substantially the entire length of the needle electrode, leaving only the distal tip exposed for electrical contact with target tissue.

The polyamide tubing may provide a thin-walled insulation barrier that minimizes the overall diameter of the needle electrode while maintaining effective electrical isolation. The polyamide material may offer advantages including chemical resistance, flexibility, and consistent wall thickness that enable reliable insulation performance throughout the PFA procedure. The Microlumen materials may provide similar insulation properties with enhanced precision in inner and outer diameter tolerances, potentially improving the fit and performance of the needle electrode within the catheter system.

Electrical connecting wires may extend through the catheter to provide electrical connectivity between the grounding electrode and needle electrode and an external controller. The electrical connecting wires may be constructed from high current capacity materials including copper, aluminum, silver, or gold to accommodate the electrical demands of PFA procedures. The copper wires may provide cost-effective conductivity with good mechanical properties for catheter applications. The aluminum wires may offer reduced weight while maintaining adequate current carrying capacity for specific applications.

The silver or gold electrical connecting wires may provide enhanced conductivity and corrosion resistance for applications requiring superior electrical performance or extended device longevity. The electrical connecting wires may be insulated along their length to prevent electrical contact with the catheter structure or other components during operation. The wires may be routed through dedicated lumens or channels within the catheter to maintain proper positioning and prevent interference with electrode deployment mechanisms.

A PFA system may operate through coordinated interaction of multiple components to achieve controlled tissue ablation while minimizing procedural invasiveness and unwanted physiological effects. The system may comprise a grounding electrode, a needle electrode, a catheter, and a controller that work together in a sequential workflow to deliver electrical energy to target tissues. The interaction between these components may enable precise electrode positioning and controlled electrical delivery while reducing muscle stimulation compared to conventional external grounding pad systems.

The operational workflow may begin with percutaneous catheter insertion, where the catheter containing the grounding electrode in a contracted state is introduced into the subject through a small incision or natural body opening. The catheter may be navigated to the target anatomical site under imaging guidance, with the grounding electrode remaining in the contracted configuration during transit through the catheter lumen. The percutaneous insertion may enable minimally invasive access to internal organs and tissues while maintaining the grounding electrode in a stable position within the catheter until deployment is initiated.

Following catheter positioning, the grounding electrode deployment phase may commence with ejection of the grounding electrode from the distal end of the catheter. The grounding electrode may transition from the contracted state to an expanded state upon exiting the catheter, with shape memory material properties causing the grounding electrode to return to a predetermined expanded configuration. The expanded grounding electrode may establish direct contact with the surface of the target tissue, creating an electrical interface that enables current flow during subsequent PFA procedures.

The tissue contact established by the grounding electrode may provide distributed electrical connectivity across the tissue surface, with the expanded configuration conforming to anatomical contours to maintain consistent electrical coupling. The grounding electrode may apply gentle pressure against the tissue surface to ensure reliable electrical contact while minimizing tissue trauma. The surface contact may eliminate the need for external grounding pads placed on the skin, reducing the electrical path length and associated impedance that can contribute to unwanted muscle stimulation in distant tissues.

Needle electrode positioning may occur after the grounding electrode has achieved stable tissue contact and full expansion. The needle electrode may be advanced through an aperture of the grounding electrode or positioned through independent insertion pathways depending on the specific system configuration. The needle electrode may penetrate into the target tissue to a predetermined depth, establishing the second electrical contact point for the PFA circuit. The positioning may maintain appropriate spacing between the grounding electrode and needle electrode to create optimal electrical field distribution within the target tissue volume.

The controller may coordinate the electrical delivery phase by generating predetermined voltage and current waveforms between the grounding electrode and needle electrode. The controller may apply an electric potential difference that creates an electrical field in the tissue region between the electrodes, inducing PFA in cells within the treatment zone. The electrical field strength may be sufficient to cause irreversible membrane permeabilization while remaining localized to the target tissue area due to the proximity of the electrodes.

The reduced distance between the grounding electrode and needle electrode may provide advantages in minimizing muscle stimulation compared to external grounding pad systems. In conventional systems where grounding pads are placed on the skin surface, electrical current may travel through extensive tissue pathways, potentially affecting nerve and muscle tissues distant from the target site. The percutaneous placement of the grounding electrode in proximity to the target tissue may confine the electrical field to a more localized region, reducing current flow through intervening tissues and associated muscle contractions.

The localized electrical field created by the proximate electrode configuration may enable the controller to achieve effective tissue ablation with lower overall current levels compared to external grounding systems. The reduced current requirements may further minimize unwanted physiological effects while maintaining therapeutic efficacy within the target tissue. The controller may monitor electrical parameters during the procedure to ensure consistent delivery and may adjust waveform characteristics based on tissue impedance measurements and treatment response.

During the electrical delivery phase, the grounding electrode may maintain stable tissue contact while the needle electrode remains positioned within the target tissue. The electrical field generated between the electrodes may create an ablation zone with predictable geometry based on the electrode spacing and applied voltage levels. The ablation zone may extend radially outward from the needle electrode toward the grounding electrode, with the field strength decreasing with distance from the electrodes according to established PFA principles.

The controller may deliver multiple electrical pulses in sequence, with controlled timing intervals between pulses to allow for tissue response and thermal management. The pulse sequences may be optimized for specific tissue types and treatment objectives, with the controller providing adjustable parameters including pulse duration, amplitude, frequency, and polarity. The electrical delivery may continue until predetermined treatment endpoints are achieved, as determined by impedance monitoring, imaging feedback, or protocol-specified pulse counts.

Following completion of the electrical delivery phase, the system components may be retracted in a controlled sequence to minimize tissue trauma and ensure complete device removal. The needle electrode may be withdrawn first, followed by retraction of the grounding electrode back into the catheter lumen. The grounding electrode may transition from the expanded state back to a contracted configuration as the grounding electrode is drawn into the catheter, with the shape memory material properties enabling the transition without permanent deformation.

The catheter may then be withdrawn from the subject through the percutaneous access site, completing the procedure with minimal tissue disruption. The sequential retraction process may ensure that all system components are removed cleanly without leaving residual materials or causing unnecessary tissue damage. The percutaneous access site may require only minimal closure procedures due to the small incision size enabled by the catheter-based delivery approach.

The coordinated interaction of the system components may enable reproducible treatment outcomes with reduced procedural complexity compared to multi-electrode systems requiring separate insertion pathways. The integrated catheter delivery may streamline the procedure workflow while maintaining precise control over electrode positioning and electrical delivery parameters. The system may provide consistent electrode spacing and orientation across multiple procedures, potentially improving treatment reproducibility and clinical outcomes for various tissue ablation applications.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A pulse field ablation system, comprising:
a grounding electrode configured for percutaneous placement in a subject;
a needle electrode configured to be positioned in proximity to the grounding electrode; and
a controller configured to apply a potential difference between the grounding electrode and the needle electrode to ablate tissue in the subject.

2. The system of claim 1, wherein the grounding electrode comprises a shape memory material.

3. The system of claim 2, wherein the shape memory material is nitinol.

4. The system of claim 1, wherein the grounding electrode comprises a substantially circular center portion and a plurality of leaflets circumferentially disposed around the center portion.

5. The system of claim 4, wherein the grounding electrode comprises an aperture extending through the center portion, and wherein the needle electrode is configured to extend through the aperture.

6. The system of claim 1, further comprising a catheter having a lumen, wherein the grounding electrode is configured to be disposed in the catheter in a contracted state and to transition to an expanded state when ejected from the catheter.

7. The system of claim 6, wherein the needle electrode is configured to be disposed within the lumen of the catheter.

8. A method of performing pulse field ablation, comprising:
placing a grounding electrode percutaneously in a subject;
positioning a needle electrode in proximity to the grounding electrode; and
applying an electric potential difference between the grounding electrode and the needle electrode to ablate tissue in the subject.

9. The method of claim 8, wherein placing the grounding electrode percutaneously in the subject comprises placing the grounding electrode on a surface of tissue to be ablated.

10. The method of claim 8, wherein the grounding electrode comprises a shape memory material.

11. The method of claim 10, wherein the shape memory material is nitinol.

12. The method of claim 8, wherein placing the grounding electrode percutaneously in the subject comprises:
providing the grounding electrode in a contracted state in a catheter;
percutaneously inserting the catheter into a body of the subject; and
ejecting the grounding electrode from the catheter, wherein upon ejection, the grounding electrode transitions from the contracted state to an expanded state.

13. The method of claim 12, wherein the grounding electrode comprises an aperture, and wherein positioning the needle electrode in proximity to the grounding electrode comprises positioning a distal end of the needle electrode through the aperture.

14. The method of claim 13, wherein the needle electrode is provided within the catheter and ejected from the catheter through the aperture of the grounding electrode.

15. A pulse field ablation device, comprising:
a catheter having a lumen;
a grounding electrode disposed within the lumen in a contracted state and configured to transition to an expanded state when ejected from the catheter percutaneously in a subject; and
a needle electrode configured to be positioned in proximity to the grounding electrode when the grounding electrode is in the expanded state.

16. The device of claim 15, wherein the grounding electrode comprises a shape memory material.

17. The device of claim 16, wherein the shape memory material is nitinol.

18. The device of claim 15, wherein the grounding electrode comprises a substantially circular center portion and a plurality of leaflets circumferentially disposed around the center portion.

19. The device of claim 18, wherein the grounding electrode comprises an aperture extending through the center portion, and wherein the needle electrode is configured to extend through the aperture when the grounding electrode is in the expanded state.

20. The device of claim 19, wherein the needle electrode is disposed within the lumen of the catheter and configured to be ejected from the catheter through the aperture of the grounding electrode.
